(19) United States
(12) Patent Application Publication
(10) Pub. No.: US 2025/0005750 A1
(43) Pub. Date: Jan. 2, 2025

(54) DETERMINING CONDUCTIVITIES OF MEDICAL IMAGES BASED ON MEASURED RESISTANCES

(71) Applicant: Novocure GmbH, Root D4 (CH)

(72) Inventors: Brian BERGER, New York, NY (US); Nadav SHAPIRA, Haifa (IL)

(73) Assignee: Novocure GmbH, Root D4 (CH)

(21) Appl. No.: 18/745,184

(22) Filed: Jun. 17, 2024

Related U.S. Application Data
(60) Provisional application No. 63/524,511, filed on Jun. 30, 2023.

(51) Int. Cl. G06T 7/00; G06T 7/12

(52) U.S. Cl. CPC G06T 7/0012; G06T 7/12; G06T 2207/20081; G06T 2207/20101; G06T 2207/30096

(57) ABSTRACT

A method for generating at least one transducer location for delivering tumor treating fields to a subject is provided. The method includes obtaining a medical image of a subject, the medical image having a plurality of voxels, the medical image representing a plurality of tissue types of the subject, wherein at least one voxel is associated with each tissue type. The method further includes determining, using a trained machine learning model and the medical image of the subject, conductivities for the tissue types of the subject in the medical image, the trained machine learning model trained with medical images of a plurality of other subjects and resistances obtained from the application of tumor treating fields to the other subjects. The method further includes identifying a location of a tumor in the medical image and generating at least one transducer location for delivering tumor treating fields to the subject.

### CROSS-REFERENCE TO RELATED APPLICATIONS

This Application claims priority to U.S. Provisional Application No. 63/524,511 filed Jun. 30, 2023, the contents of which is incorporations by reference herein in its entirety.

### BACKGROUND

Tumor treating fields (TTFields) are low intensity alternating electric fields within the intermediate frequency range (for example, 50 kHz to 1 MHZ), which may be used to treat tumors as described in U.S. Pat. No. 7,565,205. TTFields are induced non-invasively into the region of interest by transducers placed on a subject's body (i.e., the patient), and applying alternating current (AC) voltages between the transducers. Conventionally, a first pair of transducers and a second pair of transducers are placed on the subject's body. AC voltage is applied between the first pair of transducers for a first interval of time to generate an electric field with field lines generally running in the front-back direction. Then, AC voltage is applied at the same frequency between the second pair of transducers for a second interval of time to generate an electric field with field lines generally running in the right-left direction. The system then repeats this two-step sequence throughout the treatment.

### BRIEF DESCRIPTION OF THE DRAWINGS

FIG. 1 depicts an example method for training a machine learning model to determine conductivities in a medical image according to one or more embodiments described herein.

FIG. 2 depicts an example method for determining conductivities of medical images based on measured resistances according to one or more embodiments described herein.

FIG. 3 depicts an example apparatus to apply alternating electric fields to the subject's body according to one or more embodiments described herein.

FIG. 4A and 4B depict schematic views of exemplary designs of a transducer for applying alternating electric fields according to one or more embodiments described herein.

FIG. 5 depicts an example placement of transducers on a subject's head according to one or more embodiments described herein.

FIG. 6 depicts an example computer apparatus according to one or more embodiments described herein.

### DESCRIPTION OF EMBODIMENTS

This application describes exemplary techniques for training a machine learning model to predict conductivity measurements for different tissue types in a medical image and using the trained machine learning model to identify locations on a subject's body to place transducers for applying TTFields.

In general, one or more pairs of transducers are positioned on the subject's body and used to alternately apply AC voltage (e.g., TTFields) to the subject's body. Generally, it is preferred that there are at least two pairs of transducers that are arranged to target a specific location or structure (e.g., a tumor) within the subject. Accordingly, proper placement of transducers is important for treating the subject. To provide a subject with an effective TTFields treatment, precise locations at which to place the transducers on the subject's body must be generated, and these precise locations are based on, for example, the type of the cancer, the size of the cancer, and the location of the cancer in the subject's body. However, determining these precise locations is challenging, and this determination is typically accomplished using computer simulations of numerous possible locations to place the transducers on a three-dimensional model of a subject. Deriving the precise locations are time consuming and intensive.

Conventional treatment planning uses a series of measurements taken from a subject's magnetic resonance imaging (MRI) scans to measure aspects of the subject (e.g., the subject's head size, a location of the tumor, a size of the tumor, and/or the like including combinations and/or multiples thereof). Using these measurements, a customized layout for the transducers is generated. Generating transducer layouts requires considering electrical properties, such as conductivity, for the tissue types of the subject. Health care providers need to identify the various tissue types by hand, which is very tedious and can take a significant amount of time due to the large number of voxels in medical images. Once the tissue types of the subject are determined for the medical images of the subject, each voxel in each medical image has a tissue type assigned to the voxel, and conductivities can then be assigned to each of the tissue types for each of the voxels.

The inventors have now recognized that a need exists for determining conductivities of tissue types in medical images of a subject. The inventors have further discovered that measured resistances from other subjects can be used to predict conductivities of tissue types for another subject.

Embodiments described herein provide for determining conductivities for tissue types of a subject in a medical image by using a model trained with medical images of other subjects and resistances obtained from the application of TTFields to the other subjects. For example, a medical image of a subject is obtained and a trained model (e.g., a machine learning model) is used to determine conductivities for the tissue types of the subject in the medical images. A location of a tumor in the medical image of the subject can be identified and, based on the conductivities for the tissue types of the subject in the medical image and the location of the tumor in the medical image, transducer locations can be generated for delivering TTFields to the subject.

Embodiments described herein also provide for training a model (e.g., a machine learning model) to identify conductivities in medical images. For example, multiple medical images for multiple subjects are obtained, where the medical images include multiple tissue types for the subject. Measured resistances for each subject can be obtained from the application of TTFields. The measured resistances and the multiple medical images can then be used to train a model to determine conductivities in medical images. The resulting model can be used to generate transducer locations for delivering TTFields to a particular subject using the medical images for that subject.

Embodiments described herein provide for determining conductivities for tissue types of a subject in a medical image without obtaining or using electrical conductivity or resistivity measurements of the subject. Instead, for embodiments described herein, measured resistances from the application of TTFields to other subjects are obtained and used to train a machine learning model, along with medical images of the subjects, and the trained machine learning model is used to determine conductivities for tissue types in a medical image of a subject.

Embodiments described herein provide for determining conductivities for tissue types of a subject in a medical image without segmenting tissue types in the medical image of the subject to determine the conductivities. Instead, for embodiments described herein, a trained machine learning model is used to determine conductivities for tissue types in a medical image of a subject, where the machine learning model is trained using medical images of other subjects and measured resistances from the application of TTFields to these other subjects. In some embodiments, while segmenting tissue types in the medical image of the subject may be used to identify a location of a tumor or a region of interest in the subject, segmenting tissue types in the medical image of the subject does not need to be performed to determine the conductivities for tissue types in the medical image of the subject.

The embodiments described herein further provides a practical application to generate a transducer location for delivering TTFields to a subject by using the trained model to determine conductivities for tissue types of the subject using medical images for the subject. By using the medical images, such as MRI medical images and/or computerized tomography (CT) medical images, the subject's tissue conductivity is considered when generating transducer layouts for treating the subject. This leads to improved subject response to treatment because the treatment can be tailored to the subject based on the conductivities of tissue types of the subject. In some embodiments, using a trained model to determine conductivities for the tissue types of the subjects may lead to the technical advantages of generating a transducer location for delivering TTFields for the subject medical images (e.g., MRI and CT scans) of the subject to efficiently reduce the computational time needed to determine where to place transducers on the subject to deliver corresponding TTFields dosages to the subject. For example, the conductivities of tissue types can be determined using the trained model instead of manually determining tissue types by a user by reviewing the medical images of the subject. This results in faster and more accurate generation of transducer location(s). These and other technical improvements may be realized using the one or more embodiments described herein.

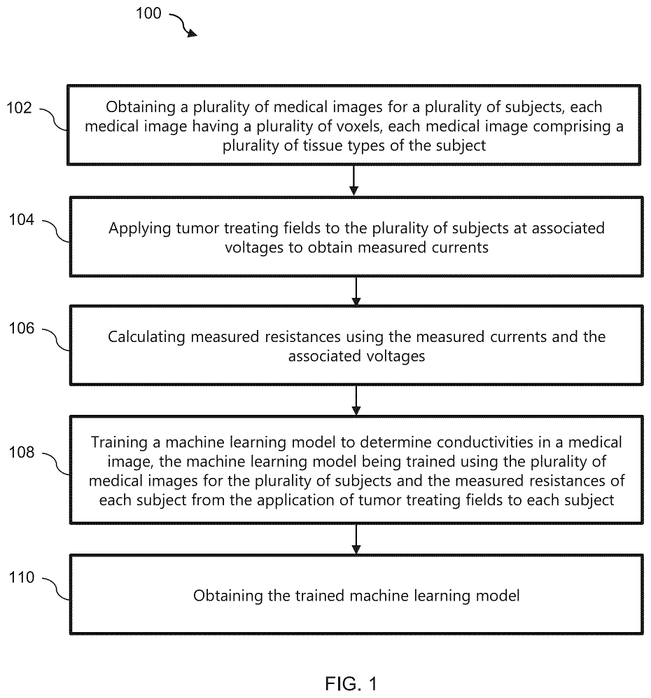

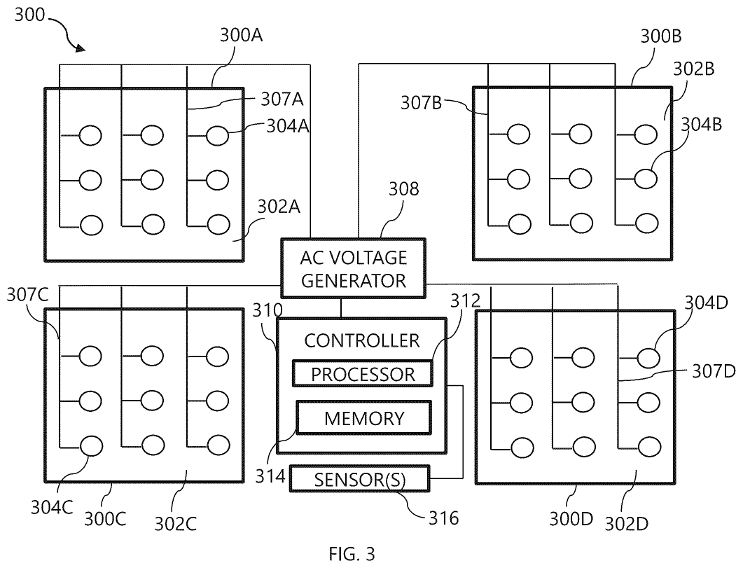

FIG. 1 is a flowchart depicting a method 100 for training a machine learning model to determine conductivities in a medical image. The method 100 can be implemented by any suitable system or apparatus, such the system of FIG. 3 and/or the apparatus of FIG. 6. While an order of operations is indicated in FIG. 1 for illustrative purposes, the timing and ordering of such operations may vary where appropriate without negating the purpose and advantages of the examples set forth in detail herein.

At block 102, the method 100 includes obtaining a plurality of medical images for a plurality of subjects, each medical image having a plurality of voxels, each medical image including a plurality of tissue types of the subject. The medical images may, for example, include at least one of a MRI image, a CT image, an X-ray image, an ultrasound image, nuclear medicine image, positron-emission tomography (PET) image, arthrogram images, myelogram images, or any image of the subject's body providing an internal view of the subject's body. Each image may include an outer shape of a portion of the subject's body and a region corresponding to a region of interest (e.g., tumor) within the subject's body. In one example, the medical image may be a three-dimensional (3D) MRI image. In an embodiment, two or more of the medical images are associated with a similar region of a subject of the plurality of subjects. The plurality of medical images can be obtained from a memory (e.g., memory 314 of FIG. 3, memory 603 of FIG. 6).

At block 104, the method 100 includes applying TTFields to the plurality of subjects at associated voltages to obtain measured currents. The measured currents are obtained for each of the plurality of subjects. As described herein, one or more pairs of transducers are positioned on a subject's body and are used to alternately apply TTFields to the subject's body.

According to one or more embodiments described herein, the measured currents can be obtained by applying the TTFields to the subjects (block 104) prior to obtaining the medical images for the subjects (block 102). In other embodiments, the medical images can be obtained (block 102) prior to obtaining the measured currents by applying the TTFields to the subjects (block 104).

At block 106, the method 100 includes calculating measured resistances using the measured currents and the associated voltages that were applied to the subjects. The measured resistances can be stored and/or retrieved from a memory (e.g., memory 314 of FIG. 3, memory 603 of FIG. 6). The measured currents can be obtained from locations on the subject(s) receiving TTFields. The measured resistances can be associated with a range of voltages and a range of frequencies from the application of TTFields to the plurality of subjects. According to one or more embodiments described herein, the currents used to calculate the measured resistances are between approximately 0.1 amps and 2.0 amps. According to one or more embodiments described herein, the currents used to calculate the measured resistances are between approximately 0.5 amps and 1.0 amps. Other ranges of currents can be implemented in other embodiments. The associated voltages can be generated by a voltage generator (e.g., voltage generator 308 of FIG. 3) used to generate TTFields. According to one or more embodiments described herein, the MRI medical images can be used to evaluate the resistance for specific electrode positions on the subject. Results of the evaluation can be compared with real resistance measurements that are acquired using the same electrode position(s), which can occur prior to or subsequent to obtaining the MRI medical images. If needed, the calculated resistances may be recomputed to improve the accuracy of the results.

At block 108, the method 100 includes training a machine learning model to determine conductivities in a medical image, the machine learning model being trained using the plurality of medical images for the plurality of subjects and the measured resistances of each subject from the application of TTFields to each subject. For example, an untrained machine learning model or model form can be trained using training data. The training data may include the plurality of medical images for the plurality of subjects and the measured resistances of each subject from the application of TTFields to each subject. As an example, a training engine can receive the training data and a model form. The model form may represent a base model that is untrained. The model form can have preset weights and biases, which can be adjusted during training. The training can be supervised learning, semi-supervised learning, unsupervised learning, reinforcement learning, and/or the like, including combinations and/or multiples thereof. The training may be performed multiple times (referred to as “epochs”) until a suitable model is trained.

The machine learning model may be a software program stored in a memory (e.g., the memory 603 in FIG. 6) and executable by a processor (e.g., one or more processors 602 in FIG. 6). The medical images may be stored in the memory and accessible by the machine learning model.

The machine learning model may take several different forms (e.g., neural network, linear regression, decision tree, support vector machine, etc.). The machine learning model may be a combination of hardware and/or software. The machine learning model may be a software program stored in memory. In some embodiments, the machine learning model may be a neural network, such as a convolutional neural network or a recurrent neural network. Example convolutional neural network algorithms used to train the convolutional neural network may include AlexNet, ResNet, or GoogLeNet, among other possibilities. Example recurrent neural network algorithms to train the recurrent neural network may include a Hopfield bidirectional associative memory network, a long short-term memory network, or a recurrent multilayer perceptron network, among other possibilities. Training machine learning models can involve minimizing a loss function. For example, the loss function for training can be based on the resistive predictions and real-world measurements (e.g., measured voltage and current at a particular frequency, where the resistivity is calculated using the measured voltage and current). According to one or more embodiments described herein, the loss function can be any suitable loss function, such as a mean square error (MSE) loss function or a mean absolute error (MAE) loss function, although it should be understood other loss functions are also possible. Once trained, the trained machine learning model is able to determine conductivities for voxels in a medical image associated with tissue of a subject.

According to one or more embodiments described herein, the machine learning model can be trained on a single channel (e.g., a pair of electrodes), on two channels (e.g., two pairs of electrodes), and/or on more than two channels (e.g., three pairs of electrodes). As an example, the machine learning model is trained on a single channel for accessing the conductivities, which can provide a cleaner measurement than training the machine learning model on multiple channels. In an example in which the machine learning model is trained on two channels, the two channels are roughly orthogonal to one another and the current through the tumor is maximized.

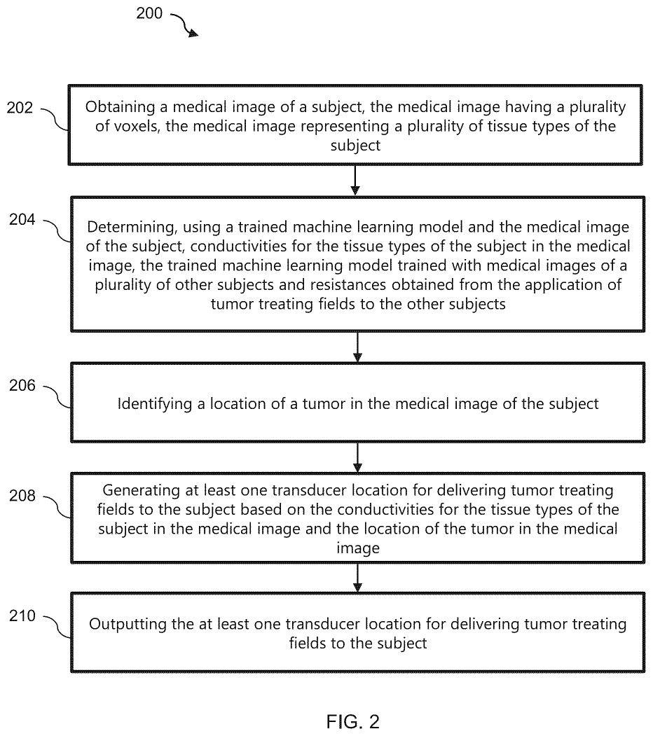

At block 110, the method 100 includes obtaining the trained machine learning model. Obtaining the trained machine learning model can include receiving, from a processing system preforming the training, the trained machine learning model. Once obtained, the machine learning model can be used to perform inference, namely to determine conductivities of medical images based on measured resistances, which is now described in more detail with respect to FIG. 2.

Particularly, FIG. 2 is a flowchart depicting a method 200 for determining conductivities of medical images based on measured resistances according to one or more embodiments described herein. The method 200 can be implemented by any suitable system or apparatus, such the system of FIG. 3 and/or the apparatus of FIG. 6. While an order of operations is indicated in FIG. 2 for illustrative purposes, the timing and ordering of such operations may vary where appropriate without negating the purpose and advantages of the examples set forth herein.

At block 202, the method 200 includes obtaining a medical image of a subject, the medical image having a plurality of voxels, the medical image representing a plurality of tissue types of the subject. According to one or more embodiments described herein, the plurality of tissue types includes one or more of skin, bone, skull, organ, brain, and/or any other tissue in the human body including combinations and/or multiples thereof. According to one or more embodiments described herein, data within the medical images can be correlated to tissue type (and eventually conductivity) using segmentation, voxel intensity, relative location within the body, and/or the like including combinations and/or multiples thereof.

At block 204, the method 200 includes determining, using a trained machine learning model and the medical image of the subject, conductivities for the tissue types of the subject in the medical image, the trained machine learning model trained with medical images of a plurality of other subjects and resistances obtained from the application of TTFields to the other subjects as described herein. Determining conductivities for the tissue types of the subject can produce a conductivity mapping of the medical image of the subject. Determining conductivities for the tissue types of the subject can be dependent on one or more frequencies to be used for delivering TTFields to a subject. The resistances obtained from the application of TTFields can be based on currents measured from applying TTFields to the other subjects at associated voltages. That is, the currents and associated voltages can be used to calculate the resistances. The trained machine learning model can be trained over a range of voltages and over a range of frequencies for the application of TTFields to the other subjects. According to one or more embodiments described herein, the trained machine learning model is for determining conductivities for a designated TTFields frequency and a designated cancer.

According to one or more embodiments described herein, the trained machine learning model is for determining conductivities for a designated frequency range for delivering TTFields. For example, the trained machine learning model is for determining conductivities for a frequency for delivering TTFields of approximately 250 kHz. As another example, the trained machine learning model is for determining conductivities for a frequency for delivering TTFields of approximately 500 kHz. It should be appreciated that these frequencies are merely examples and that the trained machine learning model can be for determining conductivities for other frequencies also or instead. Although determining the conductivities is described as being performed using a trained machine learning model, it should be appreciated that conductivities can be determined in other ways, such as manually by a user. In the case of using machine learning, the method 200 can include training a machine learning model to obtain the trained machine learning model. The machine learning model can be trained with the medical images of the other subjects and the resistances obtained from the application of TTFields to the other subjects where at least one medical image of the other subjects includes a tumor.

At block 206, the method 200 includes identifying a location of a tumor in the medical image of the subject. Identifying the location of the tumor in the medical image of the subject can be based on user input. For example, the user (e.g., a health care provider) can indicate a location of the tumor in one or more medical images of the subject. Identifying the location of the tumor in the medical image of the subject can be based on segmenting tumor tissue from other tissue in the medical image. For example, the tumor tissue can be automatically and/or manually segmented from other tissues.

At block 208, the method 200 includes generating at least one transducer location for delivering TTFields to the subject based on the conductivities for the tissue types of the subject in the medical image and the location of the tumor in the medical image. According to one or more embodiments described herein, an optimized transducer location layout can be generated at block 208. Non-limiting examples of an optimized layout include maximizing an electric field, maximizing a power density in the tumor, and/or the like including combinations and/or multiples thereof.

At block 210, the method 200 includes outputting the at least one transducer location for delivering TTFields to the subject. The transducer location(s) are locations on the subject where the transducers are to be placed for applying TTFields to the subject. For example, FIG. 5 shows a possible output transducer location having an arrangement of four transducers 500.

FIG. 3 depicts an example system 300 to apply alternating electric fields (e.g., TTFields) to the subject's body according to one or more embodiments described herein. The system may be used for treating a target region of a subject's body with an alternating electric field. In an example, the target region may be in the subject's brain, and an alternating electric field may be delivered to the subject's body via two pairs of transducer arrays positioned on a head of the subject's body (such as, for example, in FIG. 5, which has four transducers 500). In another example, the target region may be in the subject's torso, and an alternating electric field may be delivered to the subject's body via two pairs of transducer arrays positioned on at least one of a thorax, an abdomen, or one or both thighs of the subject's body. Other transducer array placements on the subject's body may be possible.

The example apparatus 300 depicts an example system having four transducers (or “transducer arrays”) 300A-D. Each transducer 300A-D may include substantially flat electrode elements 302A-D positioned on a substrate 304A-D and electrically and physically connected (e.g., through conductive wiring 306A-D). For each substrate 304A-D, the respective electrode elements 302A-D of the substrate may be electrically connected to each other and may be physically connected (e.g., through conductive wiring 307D). For each substrate 304A-D, the respective electrode elements 302A-D of the substrate may be electrically connected to each other and may be physically connected to their respective substrate 304A-D. In an example, the electrode elements 302A-D may be controlled as a collective, such that the electrode elements 302A-D receive and execute a same instruction signal. In an example, the electrode elements 302A-D may be individually controlled, such that each electrode element may receive and execute an instruction difference from an instruction received and executed by another electrode element.

The substrates 304A-D may include, for example, cloth, foam, flexible plastic, and/or conductive medical gel. Two transducers (e.g., 300A and 300D) may be a first pair of transducers configured to apply an alternating electric field to a target region of the subject's body. The other two transducers (e.g., 300B and 300C) may be a second pair of transducers configured to similarly apply an alternating electric field to the target region.

The transducers 300A-D may be coupled to an AC voltage generator 308, and the system may further include a controller 310 communicatively coupled to the AC voltage generator 308. The controller 310 may include a computer having one or more processors 312 and memory 314 accessible by the one or more processors. The memory 314 may store instructions that when executed by the one or more processors control the voltage generator 308 to induce alternating electric fields between pairs of the transducers 300A-D according to one or more voltage waveforms and/or cause the computer to perform one or more methods disclosed herein. The controller 310 may monitor operations performed by the AC voltage generator 308 (e.g., via the processor(s) 312). One or more sensor(s) 316 may be coupled to the controller 310 for providing measurement values or other information to the controller.

The electrode elements 304A-D may be capacitively coupled. In one example, the electrode elements 304A-D are ceramic electrode elements coupled to each other via conductive wiring 307A-D. When viewed in a direction perpendicular to its face, the ceramic electrode elements may be circular shaped or non-circular shaped. In other embodiments, the array of electrode elements are not capacitively coupled, and there is no dielectric material (such as ceramic, or high dielectric polymer layer) associated with the electrode elements.

The structure of the transducers 300A-D may take many forms. The transducers may be affixed to the subject's body or attached to or incorporated in clothing covering the subject's body. The transducer may include suitable materials for attaching the transducer to the subject's body. For example, the suitable materials may include cloth, foam, flexible plastic, and/or a conductive medical gel. The transducer may be conductive or non-conductive.

The transducer may include any desired number of electrode elements (e.g., one or more electrode elements). For example, the transducer may include one, two, three, four, five, six, seven, eight, nine, ten, or more electrode elements (e.g., twenty electrode elements). Various shapes, sizes, and materials may be used for the electrode elements. Any constructions for implementing the transducer (or electric field generating device) for use with embodiments of the invention may be used as long as they are capable of (a) delivering TTFields to the subject's body and (b) being positioned at the locations specified herein. In certain embodiments, at least one electrode element of the first, the second, the third, or the fourth transducer can include at least one ceramic disk that is adapted to generate an alternating electric field. In non-limiting embodiments, at least one electrode element of the first, the second, the third, or the fourth transducer includes a polymer film that is adapted to generate an alternating field.

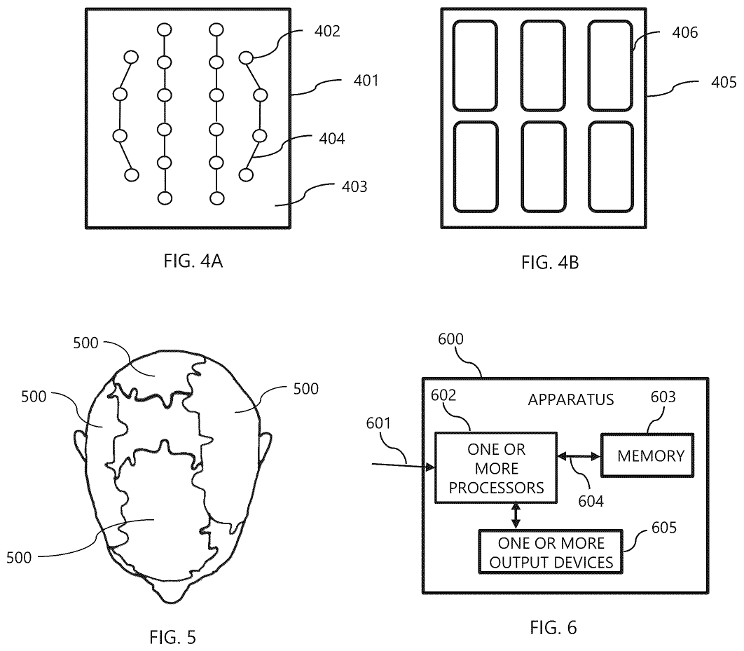

FIG. 4A depicts a schematic view of an exemplary design of a transducer for applying alternating electric fields. The transducer array 401 includes twenty electrode elements 402, which are positioned on a substrate 403, and the electrode elements 402 are electrically and physically connected to one another through a conductive wiring 404. In some embodiments, the electrode elements 402 can include a ceramic disk.

FIG. 4B depicts a schematic view of an exemplary design of a transducer for applying alternating electric fields. The transducer 405 may include one or more substantially flat electrode elements 406. In some embodiments, the electrode elements 406 and 407 are non-ceramic dielectric materials positioned over a plurality of flat conductors. Examples of non-ceramic dielectric materials positioned over flat conductors may include polymer films disposed over pads on a printed circuit board or over substantially planar pieces of metal. In an embodiment, such polymer films have a high dielectric constant, such as, for example, a dielectric constant greater than 10. In some embodiments, the electrode elements 406 can have various shapes. For example, the electrode elements can be triangular, rectangular, circular, oval, ovaloid, ovoid, or elliptical in shape or substantially triangular, substantially rectangular, substantially circular, substantially oval, substantially ovaloid, substantially ovoid, or substantially elliptical in shape. In some embodiments, each of electrode elements 406 may have a same shape, similar shapes, and/or different shapes.

FIG. 6 depicts an example computer apparatus 600 for use with the embodiments herein. As an example, the apparatus 600 may be a computer to implement certain inventive techniques disclosed herein, such as: determining conductivities of medical images based on measured resistances; generating at least one transducer location for delivering tumor treating fields to a subject; obtaining a trained machine learning model to identify conductivities in a medical image; and/or selecting transducer locations for delivering tumor treating fields to a subject. For example, blocks 102 to 110 of FIG. 1 and/or blocks 202 to 210 of FIG. 2 may be performed by a computer, such as the apparatus 600. As an example, the apparatus 600 may be a controller apparatus to apply the alternating electric fields (e.g., TTFields) with modulated electric fields for the embodiments herein. The apparatus 600 may be used as the controller 310 of FIG. 3. The apparatus 600 may include one or more processors 602, memory 603, one or more input devices, and one or more output devices 605.

In one example, based on input 601, the one or more processors 602 generate control signals to control the voltage generator to implement an embodiment of the present disclosure. In one example, the input 601 is user input. In another example, the input 601 may be from another computer in communication with the apparatus 600. The input 601 may be received in conjunction with one or more input devices (not shown) of the apparatus 600.

The memory 603 may be accessible by the one or more processors 602 (e.g., via a link 604) so that the one or more processors 602 can read information from and write information to the memory 603. The memory 603 may store instructions that when executed by the one or more processors 602 implement one or more embodiments of the present disclosure.

The one or more output devices 605 may provide the status of the operation of the invention, such as transducer array selection, voltages being generated, and other operational information. The output device(s) 605 may provide visualization data according to certain embodiments of the invention.

The apparatus 600 may be an apparatus for generating at least one transducer location for delivering tumor treating fields to a subject and/or for obtaining a trained machine learning model to identify conductivities in a medical image and/or for selecting transducer locations for delivering tumor treating fields to a subject, the apparatus including: one or more processors (such as one or more processors 602); and memory (such as memory 603) accessible by the one or more processors, the memory storing instructions that when executed by the one or more processors, cause the apparatus to perform one or more methods described herein.

The memory 603 may be a non-transitory processor readable medium containing a set of instructions thereon for generating at least one transducer location for delivering tumor treating fields to a subject and/or for obtaining a trained machine learning model to identify conductivities in a medical image and/or for selecting transducer locations for delivering tumor treating fields to a subject, wherein when executed by a processor (such as processor 602), the instructions cause the processor to perform one or more methods described herein.

### ILLUSTRATIVE EMBODIMENTS

The invention includes other illustrative embodiments (“Embodiments”) as follows.

Embodiment 1. A computer-implemented method for generating at least one transducer location for delivering tumor treating fields to a subject, the method comprising:

obtaining a medical image of a subject, the medical image having a plurality of voxels, the medical image representing a plurality of tissue types of the subject, wherein at least one voxel is associated with each tissue type; determining, using a trained machine learning model and the medical image of the subject, conductivities for the tissue types of the subject in the medical image, the trained machine learning model trained with medical images of a plurality of other subjects and resistances obtained from the application of tumor treating fields to the other subjects; identifying a location of a tumor in the medical image of the subject; and generating at least one transducer location for delivering tumor treating fields to the subject based on the conductivities for the tissue types of the subject in the medical image and the location of the tumor in the medical image.

Embodiment 2. The method of Embodiment 1, wherein determining conductivities for the tissue types of the subject produces a conductivity mapping of the medical image of the subject.

Embodiment 2A. The method of Embodiment 1, wherein determining conductivities for the tissue types of the subject is dependent on one or more frequencies to be used for delivering tumor treating fields to a subject.

Embodiment 3. The method of Embodiment 1, wherein identifying the location of the tumor in the medical image of the subject is based on user input.

Embodiment 4. The method of Embodiment 1, wherein identifying the location of the tumor in the medical image of the subject is based on segmenting tumor tissue from other tissue in the medical image.

Embodiment 5. The method of Embodiment 1, wherein the plurality of tissue types includes one or more of skin, bone, skull, organ, or brain.

Embodiment 6. The method of Embodiment 1, wherein the resistances obtained from the application of tumor treating fields are for a plurality of voltages at one or more frequencies of the tumor treating fields.

Embodiment 7. The method of Embodiment 1, wherein the resistances obtained from the application of tumor treating fields are based on currents measured from applying tumor treating fields to the other subjects at associated voltages.

Embodiment 8. The method of Embodiment 1, wherein the trained machine learning model was trained over a range of voltages and over a range of frequencies for the application of tumor treating fields to the other subjects.

Embodiment 9. The method of Embodiment 1, wherein the trained machine learning model is for determining conductivities for a designated tumor treating fields frequency and a designated cancer.

Embodiment 9A. The method of Embodiment 1, wherein the trained machine learning model is for determining conductivities for a designated frequency range for delivering tumor treating fields.

Embodiment 9B. The method of Embodiment 1, wherein the trained machine learning model is for determining conductivities for a frequency for delivering tumor treating fields of approximately 250 kHz.

Embodiment 9C. The method of Embodiment 1, wherein the trained machine learning model is for determining conductivities for a frequency for delivering tumor treating fields of approximately 500 kHz.

Embodiment 10. The method of Embodiment 1, further comprising training a machine learning model to obtain the trained machine learning model, wherein the machine learning model is trained with the medical images of the other subjects and the resistances obtained from the application of tumor treating fields to the other subjects, wherein at least one medical image of the other subjects includes a tumor.

Embodiment 11. The method of Embodiment 10, wherein training the machine learning model to obtain the trained machine learning model comprises: calculating the resistances based on currents measured from applying tumor treating fields to the other subjects at associated voltages, wherein the currents are obtained from memory.

Embodiment 11A. A method for delivering tumor treating fields to a subject, the method comprising: locating a plurality of transducer arrays on the subject, wherein at least one transducer array is located on the subject based on at least one transducer location generated by a trained machine learning model to identify conductivities in a medical image for a designated frequency and a designated cancer; and delivering tumor treating fields to the subject using the transducer arrays located on the subject.

Embodiment 12. A computer-implemented method for obtaining a trained machine learning model to identify conductivities in a medical image, the method comprising: obtaining a plurality of medical images for a plurality of subjects, each medical image having a plurality of voxels, each medical image comprising a plurality of tissue types of the subject, wherein at least one voxel in each medical image is associated with each tissue type of the subject; obtaining measured resistances of each subject from application of tumor treating fields to each subject; and training a machine learning model to determine conductivities in a medical image, the machine learning model being trained using the plurality of medical images for the plurality of subjects and the measured resistances of each subject from the application of tumor treating fields to each subject.

Embodiment 13. The method of Embodiment 12, wherein two or more of the medical images are associated with a similar region of a subject of the plurality of subjects.

Embodiment 13A. The method of Embodiment 12, wherein the plurality of medical images are obtained from memory, wherein the measured resistances are obtained from memory.

Embodiment 14. The method of Embodiment 12, wherein the measured resistances are associated with a range of voltages and a range of frequencies from the application of tumor treating fields to the plurality of subjects.

Embodiment 15. The method of Embodiment 12, further comprising calculating the measured resistances using currents measured from applying tumor treating fields to the plurality of subjects at associated voltages.

Embodiment 15A. The method of Embodiment 15, wherein the currents used to calculate the measured resistances are between approximately 0.1 amps and 2.0 amps.

Embodiment 15B. The method of Embodiment 15, wherein the currents used to calculate the measured resistances are between approximately 0.5 amps and 1.0 amps

Embodiment 16. The method of Embodiment 12, further comprising: applying tumor treating fields to the plurality of subjects at associated voltages to obtain measured currents; and calculating the measured resistances using measured currents and the associated voltages.

Embodiment 17. The method of Embodiment 16, wherein the measured currents are obtained from locations on the subject receiving tumor treating fields.

Embodiment 18. The method of Embodiment 16, wherein the associated voltages are generated by a voltage generator used to generate tumor treating fields.

Embodiment 19. The method of Embodiment 11, wherein the trained machine learning model is able to determine conductivities for voxels in a medical image associated with tissue of a subject.

Embodiment 20. An apparatus for selecting transducer locations for delivering tumor treating fields to a subject, the apparatus comprising: one or more processors; and memory accessible by the one or more processors, the memory storing instructions that when executed by the one or more processors, cause the apparatus to: determine, using a trained machine learning model and the medical image of the subject, conductivities for the tissue types of the subject in the medical image, the trained machine learning model trained with medical images of a plurality of other subjects and resistances obtained from the application of tumor treating fields to the other subjects; identify a location of a tumor in the medical image of the subject; and generate at least one transducer location for delivering tumor treating fields to the subject based on the conductivities for the tissue types of the subject in the medical image and the location of the tumor in the medical image.

Embodiment 20A. A non-transitory processor readable medium containing a set of instructions thereon that when executed by a processor cause the processor to: determine, using a trained machine learning model and the medical image of the subject, conductivities for the tissue types of the subject in the medical image, the trained machine learning model trained with medical images of a plurality of other subjects and resistances obtained from the application of tumor treating fields to the other subjects; identify a location of a tumor in the medical image of the subject; and generate at least one transducer location for delivering tumor treating fields to the subject based on the conductivities for the tissue types of the subject in the medical image and the location of the tumor in the medical image.

Optionally, for each embodiment described herein, the voltage generation components supply the transducers with an electrical signal having an alternating current waveform at frequencies in a range from about 50 kHz to about 1 MHz and appropriate to deliver TTFields treatment to the subject's body.

Embodiments illustrated under any heading or in any portion of the disclosure may be combined with embodiments illustrated under the same or any other heading or other portion of the disclosure unless otherwise indicated herein or otherwise clearly contradicted by context. For example, and without limitation, embodiments described in dependent claim format for a given embodiment (e.g., the given embodiment described in independent claim format) may be combined with other embodiments (described in independent claim format or dependent claim format).

Numerous modifications, alterations, and changes to the described embodiments are possible without departing from the scope of the present invention defined in the claims. It is intended that the present invention not be limited to the described embodiments, but that it has the full scope defined by the language of the following claims, and equivalents thereof.

What is claimed is:

1. A computer-implemented method for generating at least one transducer location for delivering tumor treating fields to a subject, the method comprising:
obtaining a medical image of the subject, the medical image having a plurality of voxels, the medical image representing a plurality of tissue types of the subject, wherein at least one voxel is associated with each tissue type;
determining, using a trained machine learning model and the medical image of the subject, conductivities for the tissue types of the subject in the medical image, the trained machine learning model trained with medical images of a plurality of other subjects and resistances obtained from an application of tumor treating fields to the other subjects;
identifying a location of a tumor in the medical image of the subject; and
generating the at least one transducer location for delivering the tumor treating fields to the subject based on the conductivities for the tissue types of the subject in the medical image and the location of the tumor in the medical image.

2. The method of claim 1, wherein determining conductivities for the tissue types of the subject produces a conductivity mapping of the medical image of the subject.

3. The method of claim 1, wherein identifying the location of the tumor in the medical image of the subject is based on user input.

4. The method of claim 1, wherein identifying the location of the tumor in the medical image of the subject is based on segmenting tumor tissue from other tissue in the medical image.

5. The method of claim 1, wherein the plurality of tissue types includes one or more of skin, bone, skull, organ, or brain.

6. The method of claim 1, wherein the resistances obtained from the application of tumor treating fields are for a plurality of voltages at one or more frequencies of the tumor treating fields.

7. The method of claim 1, wherein the resistances obtained from the application of tumor treating fields are based on currents measured from applying tumor treating fields to the other subjects at associated voltages.

8. The method of claim 1, wherein the trained machine learning model was trained over a range of voltages and over a range of frequencies for the application of tumor treating fields to the other subjects.

9. The method of claim 1, wherein the trained machine learning model is for determining conductivities for a designated tumor treating fields frequency and a designated cancer.

10. The method of claim 1, further comprising training a machine learning model to obtain the trained machine learning model,
wherein the machine learning model is trained with the medical images of the other subjects and the resistances obtained from the application of tumor treating fields to the other subjects,
wherein at least one medical image of the other subjects includes a tumor.

11. The method of claim 10, wherein training the machine learning model to obtain the trained machine learning model comprises:
calculating the resistances based on currents measured from applying the tumor treating fields to the other subjects at associated voltages, wherein the currents are obtained from memory.

12. A computer-implemented method for obtaining a trained machine learning model to identify conductivities in a medical image, the method comprising:
obtaining a plurality of medical images for a plurality of subjects, each medical image having a plurality of voxels, each medical image comprising a plurality of tissue types of the subject, wherein at least one voxel in each medical image is associated with each tissue type of the subject;
obtaining measured resistances of each subject from application of tumor treating fields to each subject; and
training a machine learning model to determine the conductivities in a medical image, the machine learning model being trained using the plurality of medical images for the plurality of subjects and the measured resistances of each subject from the application of tumor treating fields to each subject.

13. The method of claim 12, wherein two or more of the medical images are associated with a similar region of a subject of the plurality of subjects.

14. The method of claim 12, wherein the measured resistances are associated with a range of voltages and a range of frequencies from the application of tumor treating fields to the plurality of subjects.

15. The method of claim 12, further comprising calculating the measured resistances using currents measured from applying tumor treating fields to the plurality of subjects at associated voltages.

16. The method of claim 12, further comprising:
applying tumor treating fields to the plurality of subjects at associated voltages to obtain measured currents; and
calculating the measured resistances using the measured currents and the associated voltages.

17. The method of claim 16, wherein the measured currents are obtained from locations on the subject receiving the tumor treating fields.

18. The method of claim 16, wherein the associated voltages are generated by a voltage generator used to generate the tumor treating fields.

19. The method of claim 12, wherein the trained machine learning model is able to determine conductivities for voxels in a medical image associated with tissue of a subject.

20. An apparatus for selecting transducer locations for delivering tumor treating fields to a subject, the apparatus comprising: one or more processors; and memory accessible by the one or more processors, the memory storing instructions that when executed by the one or more processors, cause the apparatus to:
determine, using a trained machine learning model and medical image of the subject, conductivities for the tissue types of the subject in the medical image, the trained machine learning model trained with medical images of a plurality of other subjects and resistances obtained from an application of tumor treating fields to the other subjects;
identify a location of a tumor in the medical image of the subject; and
generate at least one transducer location for delivering the tumor treating fields to the subject based on the conductivities for the tissue types of the subject in the medical image and the location of the tumor in the medical image.
